(19) United States
(12) Patent Application Publication
(10) Pub. No.: US 2018/0325572 A1
(43) Pub. Date: Nov. 15, 2018

(54) Device, Composition and Method for Prevention of Bone Fracture and Pain

(71) Applicant: Vivek Shenoy, Redwood City, CA (US)

(72) Inventor: Vivek Shenoy, Redwood City, CA (US)

(21) Appl. No.: 16/041,310

(22) Filed: Jul. 20, 2018

Related U.S. Application Data
(63) Continuation of application No. 14/238,769, filed on Feb. 13, 2014.
(60) Provisional application No. 61/593,730, filed on Feb. 1, 2012.
(60) Provisional application No. 61/523,482, filed on Aug. 15, 2011.

(51) Int. Cl. A61B 17/88; A61L 27/52; A61B 17/00; A61B 90/00

(52) U.S. Cl. CPC A61B 17/8811; A61B 2017/005; A61B 17/8833; A61B 17/8836; A61L 27/52; A61B 2017/00942; A61B 2090/3966; A61B 2017/00495; A61B 2017/00084; A61B 2017/00022; A61B 2017/8838; A61B 2218/007; A61L 2400/06; A61L 2430/02; A61B 17/8816

(57) ABSTRACT

Methods, apparatus, compositions for reinforcing bone structures are disclosed as well as a reinforced bone structure itself. By injecting a low viscosity polymeric solution into a trabecular bone region at least partly surrounded by cortical bone allowing it to cross-link in-situ, a non-degradable gel can effectively reinforce the region by retaining fluid in the constrained space within the cortical shell. Due to the low viscosity of the pre-cross-linked aqueous polymeric solution, the entire site could be filled effectively and consistently. Additionally, by injecting a low viscosity pre-cursor, the solution fills the natural intra-trabecular spaces without substantial alteration of the trabecular structure at the site.

### RELATED APPLICATION DATA

This application is a continuation of U.S. patent application Ser. No. 14/238,769, filed Feb. 13, 2014, as the U.S. National Phase of PCT/US12/50333, filed on Aug. 10, 2012, and titled “Device, Composition and Method for Prevention of Bone Fracture and Pain”; which application claims the benefit of priority of U.S. Provisional Patent Application Ser. No. 61/593,730, filed on Feb. 1, 2012, and titled “Device, Composition and Method for Prevention of Back Pain”, and U.S. Provisional Patent Application Ser. No. 61/523,482, filed on Aug. 15, 2011, and titled “Device, Composition and Method for Prevention of Bone Fracture”. Each of these applications is incorporated by reference herein in its entirety.

### FIELD OF THE INVENTION

The present invention relates to a medical device, composition and method, and more particularly to a device, composition and method for prevention of bone fracture and pain.

### BACKGROUND

Osteoporotic fracture is a major cause of disability among the elderly. The three most common forms of osteoporotic fractures involve the proximal femur, the spinal vertebrae and the wrist. To date, most of the device approaches have focused on fixation of the fracture while prophylactic intervention to prevent fractures have involved primarily pharmaceutical approaches. Pharmaceutical approaches tend to rely on systemic drugs that can have significant side effects. As the research into screening older patients for fracture risk continues, and the ability to identify the high risk population improves, a minimally invasive prophylactic intervention targeted to the site at risk for osteoporotic fracture could have a significant impact in reducing the rate of fractures.

Osteoporotic fracture is generally attributed to be due to the loss of trabecular bone as well as the potential thinning of the cortical bone at the fracture site. Bone Mineral Density (BMD) is widely used as a diagnostic tool to assess the risk of osteoporotic fracture. Dual-energy X-ray absorptiometry (DEXA) is currently the most widely used means of measuring BMD. BMD results are reported as a T-score which is a comparison of a patient's BMD to that of a healthy thirty-year-old of the same sex and ethnicity. The criteria of the World Health Organization are a T-score of −1.0 or higher for a normal individual, between −1.0 and −2.5 for an individual with osteopenia, and −2.5 and lower for an individual with osteoporosis.

Current approaches for prophylactic intervention to prevent osteoporotic fractures in the femoral neck (femoroplasty) and the vertebrae (vertebroplasty) involve injection of bone cement (PMMA) into the trabecular bone at the site. A study evaluating femoroplasty (Sutter et al., A Biomechanical Evaluation of Femoroplasty Under Simulated Fall Conditions, J Ortho Trauma, 2010) injecting PMMA into cadaveric bone has shown that, under simulated fall conditions, injecting PMMA into the femoral neck increases fracture load and energy to fracture, and the improved mechanical performance is correlated to the level of filling of the femoral neck.

PMMA is commonly used in orthopedic surgery for reinforcing osteoporotic vertebrae as well as for filling the vertebrae after a kyphoplasty procedure. However, prophylactic use of PMMA for femoral neck fracture prevention has not gained acceptance due to the potential for bone loss due to the exothermic nature of the polymeric reaction in vivo (Heini et al., Femoroplasty—Augmentation of mechanical properties in the osteoporotic proximal femur: a biomechanical investigation of PMMA reinforcement in cadaver bones, Clin Biomech., 2004) as well as the inability to consistently fill the femoral neck/head due to the high viscosity of the polymeric mixture during extrusion into the trabecular bone. High pressures required to inject the bone cement into the trabecular bone also increase the risk of material leaking into the surrounding tissue.

Other materials like silicone and Cortoss™ (Orthovita, Malvern, Pa.), which is a cross-linked resin with glass-ceramic particles, have also been considered as potential prophylactic fillers. Recent efforts have also focused on macroporous injectable hardening resorbable calcium phosphate cements (Graftsys®, Aix-en-Provence, France) that rely on the filler material being replaced by new bone at the site.

Other methods of preventing osteoporotic fracture have relied on placing structural implants within the femoral neck (Voor et al., Device and Method to Prevent Hip Fractures, WO 2010/011855A2; Philippon et al., Femoral Neck Support Structure System and Method of Use WO2009/05883 1A1) have described a method involving the placement of an expandable mechanical structure within the femoral neck to create a cavity before injecting a filler material.

The current approaches for prophylactic treatment for fracture prevention either attempt to incorporate in-situ cross-linked materials with high compressive strength at the bony site to reinforce the surrounding cortical bone or rely on the placement of a structural implant with or without a filler material to reinforce the bone. In some of these methods, the trabecular bone structure at the site is altered during treatment.

Low back pain occurs in approximately 70-85% of all people at some time during life. Every year, a large number of new patients seek treatment for back pain. However, nearly 2 million of these patients fail to respond to current therapies. Pathology of one or more lumbar discs is felt to be the cause of low back pain in many cases. However, the origin of lumbar pain in the intervertebral disc remains a topic of wide controversy.

One of methods of assessing lumbar pain is discography. In this procedure, a radiographic contrast agent is injected into the nucleus pulposus of the disc suspected to be the source of the pain. Pain during this intra-discal injection is considered to be a confirmation of discogenic pain. However, recent studies have shown that the endplates of the adjacent vertebral bodies are deflected as a result of the intra-discal injection. These endplate deflections may cause pain sensations in the adjacent vertebral bodies, which may be the source of the pain (vertebrogenic pain).

MRI of patients with back pain are classified using a Modic scale. Type 1 changes represent bone marrow edema and inflammation. Type 2 changes are associated with conversion of normal red hemopoietic bone marrow into yellow fatty marrow as a result of marrow ischemia. Type 3 changes represent subchondral bone sclerosis.

These changes in the vertebral body are potentially caused by change in mechanical loading within the vertebral body.

Recent studies have also shown the presence of substance P within the basivertebral nerve which innervates the vertebral body. These nerves have the potential to transmit signals of nociception and may play a role in some forms of back pain.

One of the newer approaches to treating vertebrogenic pain is to ablate the basivertebral nerve within the vertebral body using radiofrequency energy. Ablation of the nerve is believed to eliminate the source of vertebrogenic pain.

### SUMMARY OF INVENTION

In exemplary embodiments of the present invention, by injecting a low viscosity polymeric solution into osteoporotic or osteopenic trabecular bone and allowing it to cross-link in-situ, a non-degradable gel can effectively reinforce bone by retaining fluid in the constrained space within the cortical shell. By injecting an in-situ cross-linking aqueous polymeric solution, a non-degradable hydrogel can effectively reinforce bone by retaining water in the constrained space within the cortical shell. The cortical shell provides an external constraint, and the polymeric hydrogel retains the water at the site. Due to the low viscosity of the pre-cross-linked aqueous polymeric solution, the entire site could be filled effectively and consistently. Additionally, unlike methods that alter the trabecular structure by creating cavities or by placing structural implants, by injecting a low viscosity pre-cursor, the solution fills the natural intra-trabecular spaces without substantial alteration of the trabecular structure at the site. In some embodiments, the polymeric precursor is injected in a substantially aqueous medium and the resulting cross-linked hydrogel retains its substantial aqueous nature.

In one implementation, the present disclosure is directed to a method for reinforcing endplates of a vertebral body, the vertebral body including a region of trabecular bone, the method comprising delivering an aqueous solution of a non-cross-linked or substantially non-cross-linked polymer into the region of trabecular bone in the vertebral body, wherein the polymer cross-links in-situ to form a non-degradable hydrogel.

In another implementation, the present disclosure is directed to a method for reducing vertebrogenic back pain, comprising delivering an aqueous solution of a non-cross-linked or substantially non-cross-linked polymer into a region of trabecular bone in a vertebral body, wherein the polymer cross-links in-situ to form a non-degradable hydrogel

In one embodiment the method for reinforcing a bone comprises delivering an aqueous solution of a non-cross-linked or substantially non-cross-linked polymer into the trabecular bone such that the polymer cross-links in-situ to form a non-degradable hydrogel in the trabecular bone.

In one embodiment the method for reinforcing a bone having a trabecular structure comprises injecting an aqueous polymeric solution into the trabecular bone such that the polymer cross-links in-situ to form a non-degradable hydrogel in the trabecular bone without substantially altering the trabecular structure at the injection site.

In one embodiment the method for reinforcing bone comprises delivering a composition into the region of trabecular bone wherein the composition is in a degradable form during delivery and transforms in-situ into a non-degradable form within the region of trabecular bone. For the purposes of this invention degradable refers to the elimination of the material from an anatomical site.

In one embodiment the injectable composition for reinforcing bone comprises a hydrophilic polymeric component with a non-degradable backbone and at least two active end-groups, and a cross-linking agent. The composition is formulated such that the cross-linked hydrogel that is formed within the trabecular bone is non-degradable under physiological conditions.

In one embodiment an aqueous non-degradable cross-linked hydrogel is formed in-situ at an intra-osseous site in osteopenic or osteoporotic bone.

In one embodiment, the cross-linked hydrogel is bio-inert.

In one embodiment, the cross-linked hydrogel has a compressive modulus substantially lower than healthy cancellous bone.

In one embodiment, the cross-linked hydrogel has compressive strength substantially lower than healthy cancellous bone.

In one embodiment the hydrogel formed in-situ at an intra-osseous site in osteopenic or osteoporotic bone comprises a polymeric backbone and cross-links that are non-degradable under physiological conditions.

In one embodiment, the treatment is directed towards the proximal femoral neck.

In one embodiment, the treatment is directed towards a vertebral body.

In one embodiment, the treatment is directed towards the humeral head.

In one embodiment, the treatment is directed towards the wrist, the site of Colles fracture.

In some embodiments, the injectable in-situ cross-linked hydrogel may contain additives that confer some compressibility to the hydrogel (i.e., poisson's ratio of less than 0.5).

In some embodiments, the treatment step includes injection of material to contain the cross-linked hydrogel at the injection site.

In some embodiments, the injection site may be evacuated before injecting the in-situ cross-linking hydrogels.

In some embodiments, the injection site may be prepared by removal of any residual non-bony tissue before injecting the in-situ cross-linked hydrogel.

In some embodiments, the composition may contain visualization agents like radio-opaque agents and dyes, thickening agents that increase the viscosity of the composition, cells, growth factors, antibiotics and other bioactive compounds.

In some embodiments, the injectable composition for reinforcing bone is radio-opaque during injection into the trabecular bone.

In some embodiments, the components of the hydrogel are provided in a sterile form with a delivery device to enable treatment of the bony site.

In some embodiments, a kit for preparation and delivery of the treatment is disclosed.

In some embodiments, a system for reinforcing bone by delivering an injectable hydrogel into an intra-osseous site is disclosed.

In one embodiment the system for reinforcing bone comprises a reservoir with an aqueous polymeric solution, a delivery tip and a pressurization device. The aqueous polymeric solution in the reservoir is formulated to become a cross-linked hydrogel when delivered within the trabecular bone. The delivery tip is configured to penetrate the cortical layer surrounding the trabecular bone at the site, and has a lumen in fluid communication with the reservoir. The pressurization device is configured to apply pressure to the reservoir to deliver the polymeric composition.

In one embodiment the kit for reinforcing bone comprises a polymeric solution, a cross-linker, a means for combining the polymeric solution and the cross-linker, a delivery device for delivering the combination of the polymeric solution and the cross-linker to the bony site. The delivery device comprises a reservoir for containing the combination of polymeric solution and the cross-linker, a pressurization device configured to apply pressure to the reservoir, and a delivery tip in fluid communication with the reservoir and configured to pass through the skin and penetrate the cortical layer into the trabecular region at the bony site.

In one embodiment the method for reinforcing the vertebral body endplates comprises evacuating the vertebral body, delivering an aqueous solution of a non-cross-linked or substantially non-cross-linked polymer into the trabecular bone such that the polymer cross-links in-situ to form a non-degradable or slowly degrading hydrogel in the trabecular bone.

In one embodiment the method for reinforcing the vertebral body endplates having a trabecular structure comprises evacuating the vertebral body, injecting an aqueous polymeric solution into the trabecular bone such that the polymer cross-links in-situ to form a non-degradable or slowly degrading hydrogel in the trabecular bone without substantially altering the trabecular structure at the injection site.

In one embodiment the method for reinforcing the vertebral body endplates comprises delivering a composition into the region of trabecular bone wherein the composition is in a degradable form during delivery and transforms in-situ into a non-degradable or slowly degrading form within the region of trabecular bone. For the purposes of this invention degradable refers to the elimination of the material from an anatomical site.

In one embodiment the injectable composition for reinforcing the vertebral body endplates comprises a hydrophilic polymeric component with a non-degradable backbone and at least two active end-groups, and a cross-linking agent. The composition is formulated such that the cross-linked hydrogel that is formed within the trabecular bone is non-degradable or slowly degradable under physiological conditions.

In one embodiment an aqueous non-degradable or slowly degrading cross-linked hydrogel is bio-inert.

In one embodiment the hydrogel formed in-situ at an intra-osseous comprises a polymeric backbone and cross-links that are non-degradable or slowly degrading under physiological conditions.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1a is a side view of a human femoral head illustrating the relative locations of trabecular bone and the cortical bone shell.

FIG. 1b is a radiograph of a femoral head showing location and structure of trabecular bone.

FIGS. 2a and 2b illustrate injection of a polymeric solution to form a cross-linked hydrogel within the trabecular structure of a femoral head according to an embodiment of the present invention.

FIGS. 3a and 3b are schematic enlargements of the trabecular bone structure illustrating, respectively, the marrow space and the marrow space filled by a cross-linked hydrogel according to an embodiment of the present invention.

FIGS. 4 and 5 are schematic illustrations of further embodiments of the present invention as applied to a femoral head.

FIG. 6a is a side view of a human vertebra illustrating the area of the trabecular bone.

FIG. 6b is a radiograph of a vertebral body showing location and structure of trabecular and cortical bone.

FIGS. 7 and 8 illustrate injection of a polymeric solution to form a cross-linked hydrogel within the trabecular structure of a vertebral body according to an embodiment of the present invention

FIG. 9 is a schematic illustration of the use of microspheres to alter the compressibility of a hydrogel in accordance with an alternative embodiment of the present invention.

FIGS. 10, 11a, 11b, 12, 13, 14, 15, 16 and 17 illustrate various injection devices for cross-linkable reinforcing liquids according to alternative embodiments of the present invention.

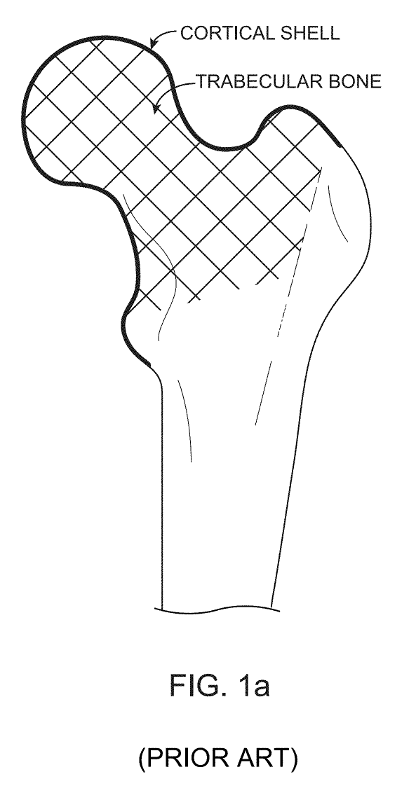

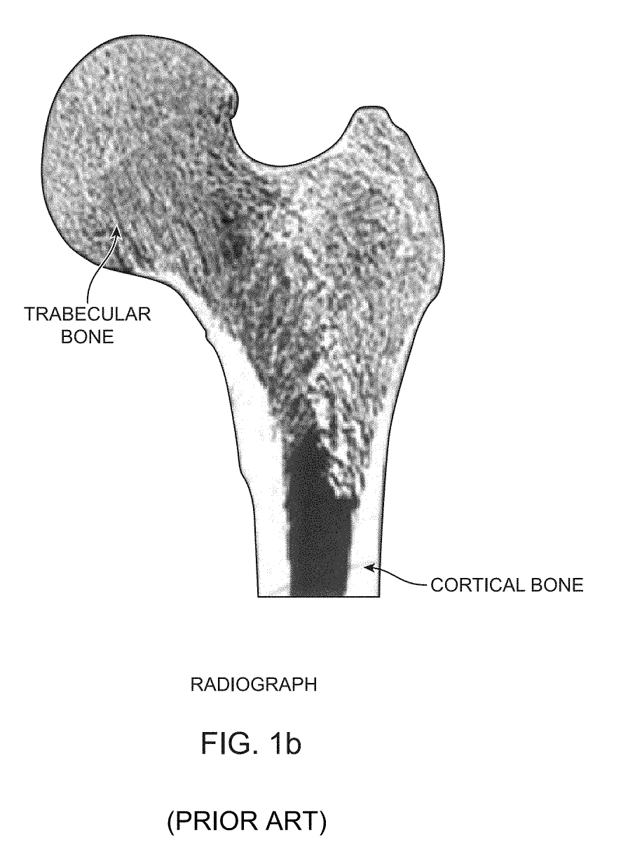

### DESCRIPTION OF EMBODIMENTS OF THE INVENTION

Embodiments of the present invention are directed towards the prevention of fractures and/or the reduction of pain attributable to a weakened state of the bone by filling targeted voids within the bone with an incompressible fluid, in the form of a stable, non-degradable, cross-linked gel, for example, a hydrogel. In one embodiment, the treatment is directed towards osteopenic or osteoporotic bone which are at greater risk of fracture. In other embodiments, the treatment is directed towards the prevention of osteogenic pain, and, in particular, towards non-specific vertebrogenic back pain. Applications include but are not limited to treatments in the femoral head and vertebral bodies. In various embodiments, including treatment methods, compositions, and apparatus, water or other suitable incompressible liquids are employed to provide mechanical support by being retained within a contained space defined by the existing bone structure. In some disclosed embodiments, a polymeric precursor is injected into trabecular bone in a substantially liquid medium and the resulting cross-linked gel reinforces the bone by retaining fluid in the constrained space within the cortical shell. Alternative embodiments include the polymeric precursor being injected in a substantially aqueous medium such that the resulting cross-linked hydrogel retains its substantial aqueous nature.

In general, terminology used herein is in a manner consistent with its ordinary use in the art. However, for the sake of clarity, the following terms are specifically defined as related to embodiments of the present invention. The term “bioactive” as used herein refers to a material that is biocompatible that interacts with or forms chemical or biological bonds with the cellular and extracellular components of tissue at the implantation site (e.g., bone, cartilage, etc.). The term “bio-inert” as used herein refers to a material that is biocompatible but cannot induce any interfacial biological bond between the material and the cellular and extracellular components of tissue at the implantation site (e.g., bone, cartilage, etc.). The term “gel” as used herein refers to a three-dimensional polymer network in a liquid medium. The term “hydrogel” as used herein refers to a three-dimensional polymeric gel in an aqueous medium. The term “incompressible” as used herein refers to a material with a poisson's ratio of substantially 0.5. The term “non-degradable” as used herein with reference to a gel refers to a gel wherein at least about 50% of the gel remains in-situ under physiological conditions after at least one year.

Target regions for treatment in embodiments of the present invention are regions of trabecular bone surrounded by cortical bone either entirely (e.g., vertebral bone) or substantially (e.g., femoral head and neck). As is well understood by persons of skill in the art, bone is generally classified into cortical bone, also known as compact bone, and trabecular bone, also known as cancellous or spongy bone. Cortical bone is found primarily in the shaft of long bones and forms the outer shell around trabecular bone at the end of joints and the vertebrae. Trabecular bone is characterized by trabecule that form spaces or voids filled with blood vessels and bone marrow. One function of trabecular bone is to provide support to the ends of the weight-bearing bone.

Indications for treatment using embodiments of the present invention will typically involve regions where the cortical shell of the target bone is substantially intact and not compromised or fractured. In such embodiments, to mechanically reinforce the cortical shell of the target bone, the strengthening material should substantially fill the target bone.

“Substantially filled” refers to at least about 75% of the inter-trabecular volume of the target bone being filled by the reinforcing gel. In certain embodiments the amount of fill will be greater than about 85% of the inter-trabecular volume of the target bone, and where possible greater than about 95% of the inter-trabecular volume of the target bone. These fill ratios are generally applicable regardless of the specific target bone region, for example, the vertebral body, femoral head or femoral head and neck. In some embodiments, as described below, the reinforcing gel is a cross-linked gel, for example, a cross-linked hydrogel.

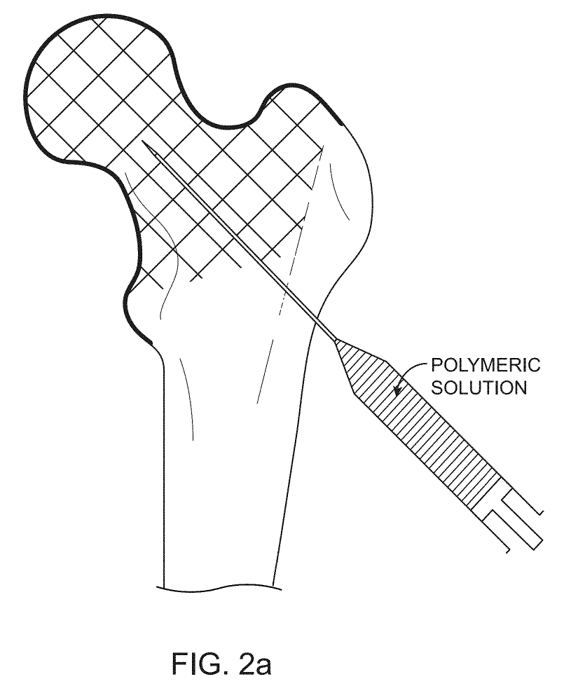

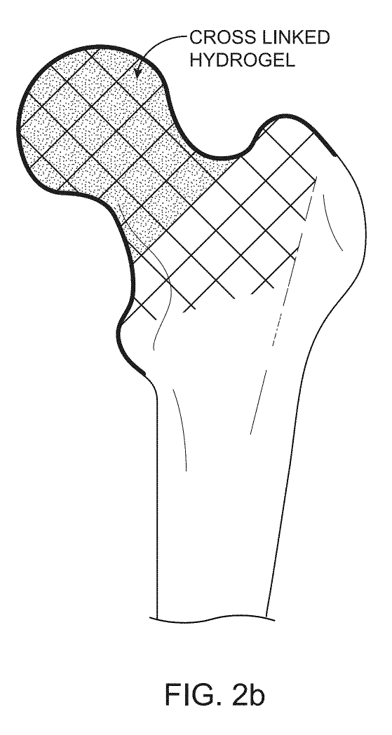

In one exemplary embodiment of the invention, an osteoporotic proximal femoral head/neck is filled by injecting a low viscosity aqueous polymeric solution through the cortical shell and into the trabecular bone within femoral head/neck as shown in FIG. 2a and allowing it to cross-link in-situ as shown in FIG. 2b. Fluid content of the femoral head/neck including red marrow, yellow marrow, fat, blood, etc. may be aspirated out prior to injecting the polymeric solution. Due to the low viscosity of the pre-cross-linked aqueous polymeric solution, it may be possible to fill the entire femoral neck/head completely and consistently. By forming a hydrogel to fill the inter-trabecular space within the femoral head/neck, the cortical shell is reinforced by the water retained in the constrained space.

Treatment in accordance with embodiments of the present invention may result in the formation of a region of reinforced bone structure characterized by a region of trabecular bone surrounded at least in part by a layer of cortical bone with a cross-linked hydrogel filling substantially more than half of the volume of the interstices defined by the region of trabecular bone. In such a reinforced bone structure, the peak load to failure of the treated bone structure (e.g., reinforced vertebral body, reinforced femur, etc.) under compressive loading may be up to about 15% greater than that of the bone prior to treatment. The amount of actual increase in strength will depend upon factors such as the integrity of the existing bone structure and the ability to achieve a fill rate at or exceeding about 75% of the volume defined by the interstices of the trabecular bone. At higher fill levels it may be possible to achieve up to about a 30% increase in strength or even in some cases up to about a 40% increase in peak load failure as compared to the untreated bone. The energy to failure ratio of the treated bone structure (e.g., reinforced vertebral body, reinforced femur) under compressive loading would be preferably about 100%, more preferably about 125%, most preferably 150% greater than that of the untreated bone; again based on the same factors.

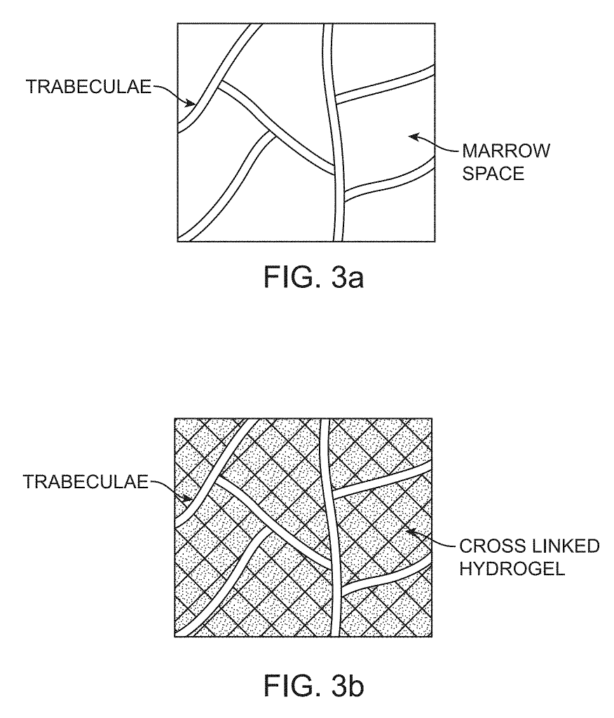

In a further embodiment, the polymeric content of the pre-cross-linked polymeric solution would be less than about 15% (by weight of the composition), more specifically less than about 10%, and in some embodiments less than about 5%. The cortical shell provides an external constraint, and the polymeric hydrogel retains the water at the site. Due to the low viscosity of the pre-cross-linked aqueous polymeric solution, it should be possible to fill the inter-trabecular space over at least substantially the entire target site. Additionally, as illustrated in FIGS. 3a and 3b, the trabecular bone structure at the site may be left essentially unaltered as a result of the treatment. In general, with embodiments of the present invention it is not necessary to alter the trabecular bone structure in the target region prior to injection of the polymeric solution (such as, e.g., by using a mechanical device to create a void within the target bone or by inflating a balloon device within the target bone). Moreover, due at least in part to the low viscosity, low pressure and relatively small injection site required for embodiments of the invention, the treatment as disclosed herein may be accomplished without altering the trabecular bone structure during or immediately after the injection of the polymeric solution. In addition, embodiments of the present invention permit the composition of the hydrogel in accordance therewith to be formulated such that it does not adversely affect the viability of the cellular components of the trabecular bone within the target region for at least 6 months, preferably for at least 9 months, most preferably for at least 12 months or more.

In another aspect, embodiments of the present invention treatment in accordance therewith may be performed under local anesthesia using fluoroscopic guidance. In one embodiment, a trocar, needle, or other suitable delivery device would be placed into the femoral neck/head as shown in FIG. 2a. If desired, any fluid or fatty tissue within the trabecular bone could be aspirated out before injecting the aqueous polymeric solution. Alternatively or additionally, the trabecular bone could be subjected to jet lavage to remove any loose tissue fragments and material loosely attached to the surface of the trabeculae and cortical shell before injecting the polymeric solution. The needle may be held at the injection site until the polymeric solution is cross-linked sufficiently. With reference to embodiments of the present invention, cross-linking as used herein refers to links formed between polymeric chains by covalent bonds, electrostatic interactions, mechanical entanglements and other means that convert the injected material from a relatively low viscosity, readily flowable liquid to a higher viscosity, gel-like state (i.e., the elastic or storage modulus G′ exceeds the loss or viscous modulus G″), and thus renders the cross-linked material at least substantially non-flowable and at least substantially non-degradable under physiological conditions.

In further alternative embodiments of the present invention, injection of the polymeric mixture with a visualization aid like a radio-opaque agent for fluoroscopic imaging may be used to provide real time feedback on the location of the polymeric mixture and to ensure that the mixture is delivered consistently to the trabecular site of interest. Radio-opaque or contrast agents may be water soluble or water insoluble.

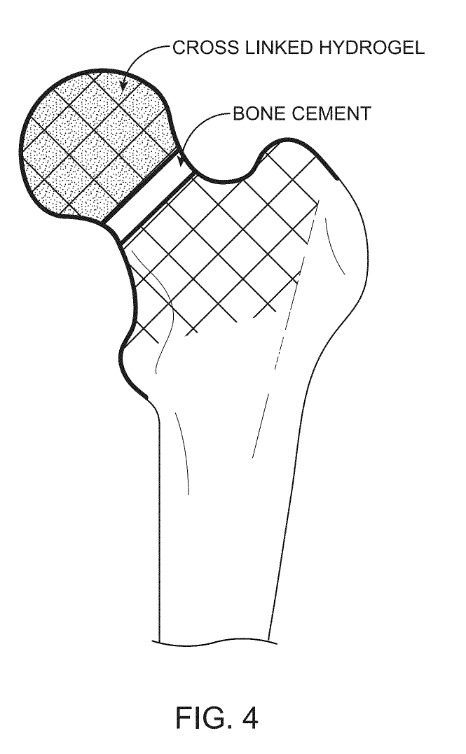

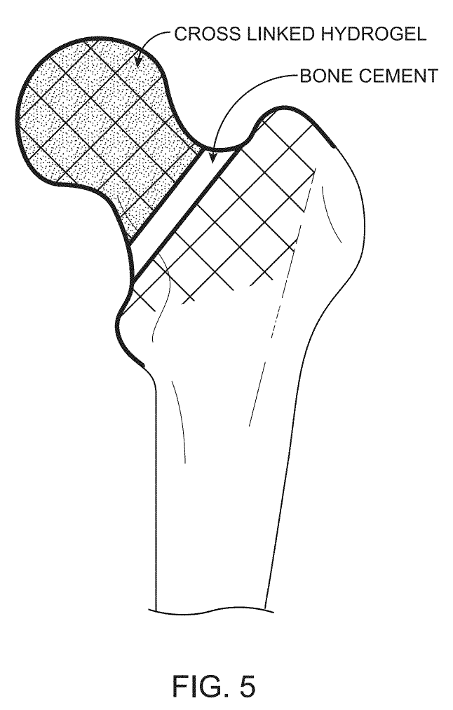

Treatments according to embodiments of the present invention may be configured by the provider in accordance with patient specific anatomical and pathological conditions. As such, the procedure may involve appropriate selection of the target region for treatment, for example, with respect to treatments of the femur, filling only the femoral head, only the femoral head and the femoral neck, or, the femoral head and neck as well as the intertrochanteric region. In certain embodiments of the invention, to define the target region and thus better contain the polymeric fluid prior to cross-linking or to retain the cross-linked hydrogel within the desired region of the bone, bone cement may be injected to form a dam or plug as shown in FIGS. 4 and 5. Such a bone cement dam may fill the intra-trabecular spaces across a transverse section of the bone and seal off the target trabecular bone region from the remainder of the trabecular bone. The bone cement may be injected prior to injecting the polymeric solution or after the polymeric solution has been injected.

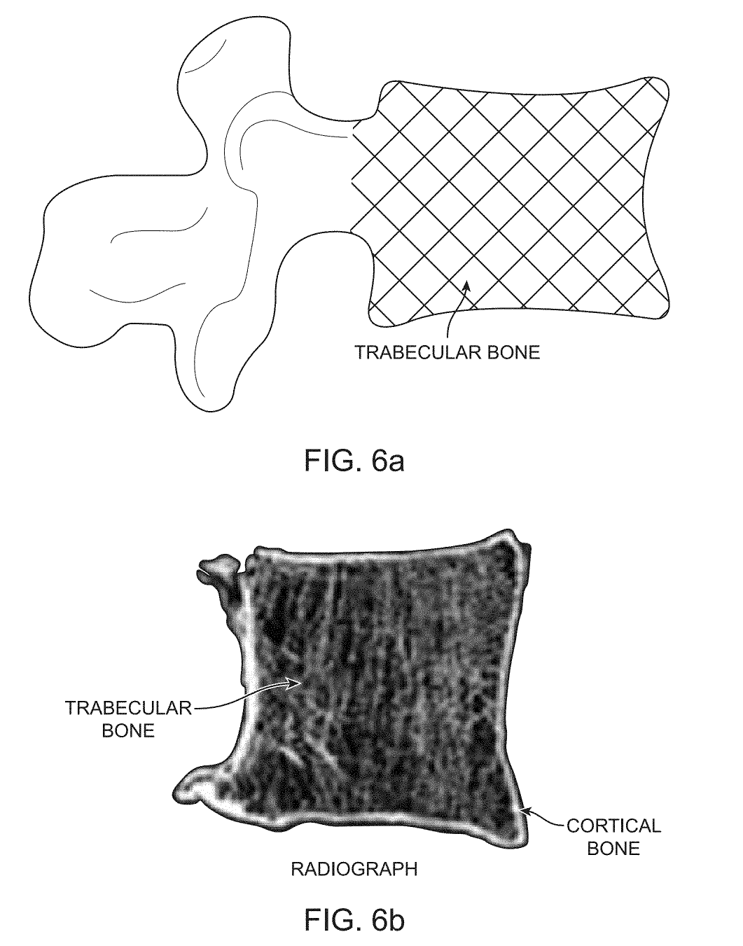

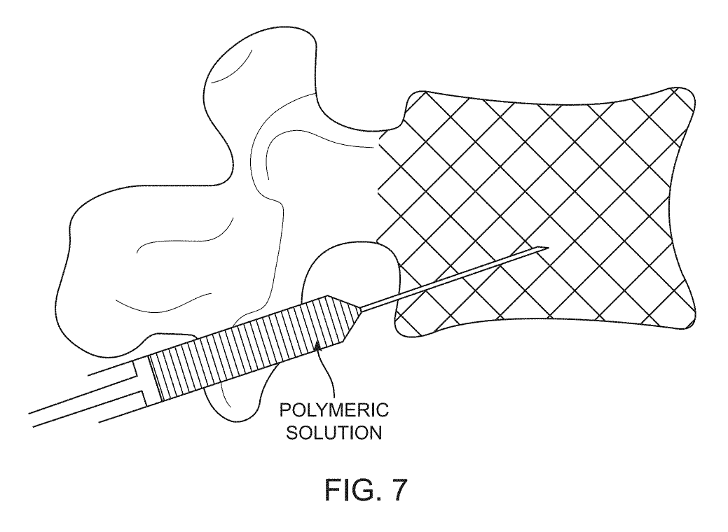

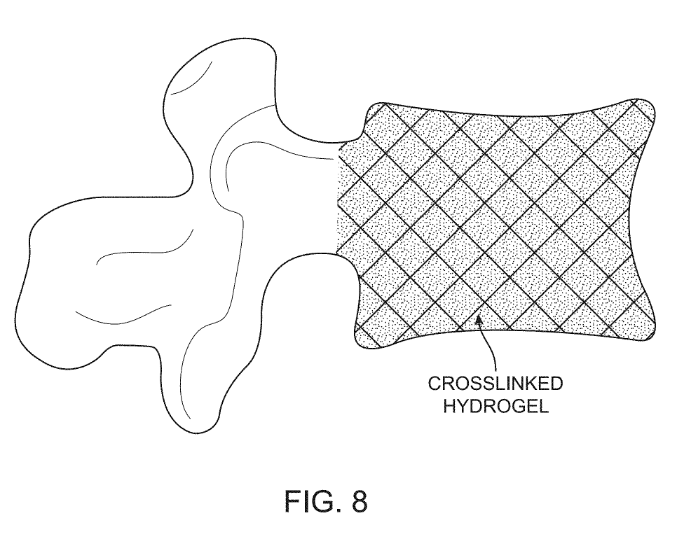

In other embodiments of the present invention, osteoporotic vertebrae, as shown in FIGS. 6a and 6b, may be filled in similar fashion as previously described. In one embodiment, as illustrated in FIG. 7, a low viscosity aqueous polymeric solution is injected into the vertebral body and, as illustrated in FIG. 8, allowed to cross-link in-situ. The fluid content of the vertebral body including red marrow, yellow marrow, fat, blood, etc. may be aspirated out prior to injecting the polymeric solution. To contain the polymeric solution within the vertebral body, it is preferable that the cortical shell corresponding to the target region is not disrupted or at least not substantially disrupted to help contain the treatment gel.

Various exemplary embodiments discussed above related to the femur and vertebrae are considered to be illustrative and not intended to limit the scope of the present invention with respect to treating other bony sites like the wrist, the humeral head, etc., which are prone to higher fracture risk due to osteoporosis or osteopenia. In general, treatments according to embodiments of the present invention may be applied in any bony structure comprising trabecular-like inner region at least partially surrounded by a relatively intact containment structure such as a cortical bone layer.

In other embodiments of the present invention, pain arising from compromised bone structures may be reduced or eliminated. One of the potential origins of pain within the vertebral body via the basivertebral nerve may be a result of mechanical stimulation of the nerve endings within the vertebral body due to endplate deflection. Changes in the composition of the vertebral, as detected by MRI, specifically near the endplates, may alter the mechanical response of the vertebral body to compressive loading. It is possible that the changes in the mechanical strength of the vertebral body may cause dynamic changes in the trabecular structure around the nerve endings, leading to neurogenic pain. By filling the vertebral body with a reinforcing gel, such as a hydrogel described in connection with embodiments of the present invention, the endplates may be reinforced, thereby reducing the deflection of the endplates under axial loading of the spine. By reducing the endplate deflection, the mechanical stimulation of the basivertebral nerve endings may be concomitantly reduced or eliminated, thereby eliminating a source of vertebrogenic pain. Reinforcing the vertebral body with a non-degradable reinforcing gel in accordance with embodiments of the present invention, may reduce endplate deflection (as measured by discography) by at least about 50%, more specifically by at least about 75%, and in some embodiments by at least about 90%. Such a reduction would be in comparison to the endplate deflection that could be detected by discography without reinforcing liquid injected in the vertebral body. The reduction in endplate deflection is measured when the injected reinforcing gel has substantially cross-linked within the vertebral body, and has not degraded substantially.

In further alternative embodiments of the invention, the composition of the reinforcing gel, such as hydrogels, may be selected to reduce the irritation of the basivertebral nerve endings, thereby providing additional pain relief. For example, the presence of the cross-linked reinforcing gel around the nerve endings may reduce the release of substance P which is released in response to nociceptive stimuli. As desired, substances having an anesthetic effect may be added to the reinforcing gel to enhance the pain relief effect in this regard.

It is within the scope of this invention, given that the cross-linked reinforcing gel may be a slowly degrading material, that the bone region targeted for treatment, whether femoral, vertebral or other suitable bone structure, may be re-injected with an in-situ cross-linking reinforcing gel after the reinforcing gel from an initial treatment has partially degraded. The decision to re-inject the vertebral body may be made based on assessment of residual cross-linked reinforcing liquid in the vertebral body (by MRI, for example) or by increase in back pain or by increase in endplate deflection during discography. As described herein, the cross-linkable reinforcing gel may comprise a hydrogel.

Based on the teachings of the present invention as set forth herein, a person of ordinary skill in the art may adapt known means of forming a cross-linked hydrogel in-situ for use in connection with embodiments of the present invention. Cross-linking may be initiated just before injection, during injection or after the material is injected into the bony site. Without being limited by theory, a non-cross-linked polymeric solution may be converted into a cross-linked hydrogel in-situ by various means like increase in temperature, free-radical reaction by exposure to energy such as visible light, UV light, x-ray, microwave, ultrasound, etc., free-radical reaction using chemical reactions, or by premixing an active cross-linker before injecting the mixture into the bony site where a substantial amount of the cross-linking occurs in-situ. Depending on the specific cross-linking modality, additional components like catalysts or inhibitors could be added to accelerate or slow down the rate of cross-linking. To reduce risk of undesirable side effects, the cross-linking reaction may be selected such that it is not exothermic and generates minimal heat during the reaction such that the temperature of the bone at the injection site is essentially unchanged during the procedure.

By way of example, and without being limited by theory or to specific chemical formulations, in-situ chemical cross-linking may be generally accomplished by vinyl-vinyl, vinyl-thiol and thiol-thiol coupling mechanisms. Vinyl-vinyl coupling may be performed via free radical polymerization, or radical-chain addition polymerization, of water-soluble compounds. For chemically-initiated free radical polymerization, a water-soluble redox initiator may be used. A common pair of redox initiators is ammonium persulfate and L-ascorbic acid. The concentration of both the oxidizer (i.e., persulfate) and reducer (i.e., ascorbate) may be altered to alter the kinetics of the reaction. Some common concentrations of the redox components are disclosed in Behravesh et al., Biomacromolecules 3, 374-381, 2002, which is incorporated by reference herein. Catalysts like FeCl₃ may be used to accelerate the cross-linking kinetics. In photopolymerization, visible or UV light irradiation may be used to generate a free radical from a compound, or photoinitiator, which has strong light absorption sensitivity at a specific wavelength. Some photoinitiators, such as acetophone derivatives and other aromatic carbonyl compounds, generate free radicals by the photocleavage of C—C, C—Cl, C—O or C—S bonds. Vinyl-thiol cross-linking occurs through a Michael-type addition reaction that results in the stepwise copolymerization of vinyl-functionalized polymer units (polyacrylates) with thiol-functionalized polymer units (e.g., polycysteines).

For most effective prophylactic benefit, after cross-linking, the reinforcing gel according to embodiments of the invention would be at least substantially non-degradable in vivo. Gels or hydrogels in various embodiments, after they are cross-linked in-situ, are at least substantially non-degradable or, in some instances, may be very slowly degradable under physiologic conditions to the extent that the treatment is effective for a sufficient period of time. In particular, polymers with backbones that are substantially resistant to physiological degradation mechanisms and not degradable or slowly degradable by various physiological mechanisms including enzymatic, radical, hydrolytic, etc., may be used. Similarly, cross-links that are substantially resistant to physiological degradation mechanisms and are not degradable or slowly degradable by various physiological mechanisms including enzymatic, radical, hydrolytic, etc., also may be used. The polymeric backbone may have at least two end-groups that are capable of forming non-degradable crosslink. In some embodiments, a branched polymeric backbone may be used with multiple end-groups capable of forming non-degradable cross-links. The polymeric backbone may have the only one type of end-group or different types of end-groups. In some embodiments, some of the end-groups may form degradable cross-links provided that there are at least two end-groups on each polymeric backbone (or branched polymer) that are capable of forming non-degradable or slowly degradable cross-links.

In further embodiments of the present invention, a polymeric pre-cursor and cross-linker can be selected to ensure that the cross-linked hydrogel is substantially non-degradable, for example, cross-linked polyethylene glycol di-acrylate (PEG-DA) hydrogels are known to be relatively resistant to degradation in vivo. Other active end-groups like methacrylate, vinyl sulfone and diacrylamide may be used. Hydrogels selected for use in embodiments of the invention should be non-degradable under physiological conditions encountered in inter-trabecular bone. In the case of low molecular weight of PEG-DA (e.g., MW<20 KDa), the viscosity of the non-cross-linked polymer solution could be relatively low, thereby enabling easy intra-osseous injection into the trabecular bone. In preferred embodiments, since the cross-linked hydrogel does not need to be inherently strong mechanically (high compressive strength and compressive modulus), the concentration of the polymer in the hydrogel can be low. Low polymer concentration confers benefits such as low viscosity during injection. Additionally, softer hydrogels formed due to low polymeric concentration may confer benefits of mechanical compliance of the reinforced bone when the cortical shell is not completely surrounding the hydrogel, for example, in the femoral head/neck as shown in FIG. 2B. Other polymers like poly-vinylpyrrolidone (PVP), poly(hydroxyethyl methacrylate), poly(vinyl alcohol), and poly(ethylene-co-vinyl acetate) may also be used with appropriate modifications to ensure their solubility in water. In exemplary embodiments, the monomers or co-monomers or macromers forming the polymeric backbone are hydrophilic, and are free of hydrophobic domains. It will be understood by persons skilled in the art based on the teachings contained herein that polymers disclosed which may have hydrophobic domains in the polymeric backbone could be modified chemically to render them substantially hydrophilic for use in the present invention. Presence of hydrophobic domains could alter the ability of the hydrogel to retain water, thereby impacting the ability of the hydrogel to reinforce the cortical shell of the target bone. Additionally, the presence of hydrophobic domains may alter the biocompatibility of the hydrogel and adversely affect the viability of the trabecular bone within the target bone. The polymeric precursor may be injected in a substantially aqueous medium and the resulting cross-linked hydrogel retains its substantial aqueous nature. Any water soluble polymeric entity with a non-degradable backbone structure, modified with end-groups that can form non-degradable cross-links, could be used in embodiments of this invention. For example, a polymer like water-soluble polyamidhydroxyure-thane as described by Melnig et al. (Melnig V. et al., Water-soluble polyamidhydroxyurethane swelling behavior, J. Optoelectronics and Adv. Mat., 2006), which is incorporated herein by reference, may be used. The examples above are exemplary and illustrative and one skilled in the art would be able to design other polymeric entities and cross-linked hydrogels that are within the scope of this invention.

Other formulations of hydrogels may be useful in alternative embodiments of the present invention. Methods of radical polymerization of hydrogels using poly(ethylene glycol) vinyl monomers (e.g., polyethylene glycol diacrylate, polyethylene glycol tetracrylate, polyethylene glycol methacrylate etc.) are described in Johnson et al., Biomacromolecules, 10, p 3114-3121, 2009. For instance thermally activated cross-linking can be accomplished by using ammonium persulfate and tetramethylethylenediamine. Alternatively, poly(vinyl alcohol) could be cross-linked using a redox initiation system comprising of a ferrous salt and hydrogen peroxide. Enzyme mediated initiation systems like glucose oxidase, glucose and a ferrous salt may also be preferred. A method of forming a PVP hydrogel using a Fenton redox reaction is disclosed in Barros et al., Polymer 47, p 8414-8419, 2006. Poly(ethylene glycol) hydrogels may also be formed in-situ by mixing polyethylene glycol-amide-succinimidyl glutarate and trilysine and injecting the mixture prior to gelation. Non-biodegradable and non-resorbable biopolymers that could be cross-linked to form non-degradable or slowly degradable gels are disclosed in Haddock et al. (US 20110182849). The entire disclosure of this published patent application, as well as the forgoing references, are incorporated by reference.

In other exemplary embodiments of the present invention, the cross-linked reinforcing gel, for example a hydrogel, is bio-inert. As defined above, a bio-inert material as used herein is a biocompatible material that does not induce any interfacial biological bond between the material and the cellular and extracellular components of tissue at the implantation site (e.g., bone, cartilage, etc.). Bioactive materials, on the other hand, when implanted in the body, form chemical or biological bonds with the cellular and extracellular components of tissue at the implantation site (e.g., bone, cartilage, etc.). Most bioactive materials tend to be bioresorbable and are eventually replaced by new tissue in vivo in less than 6 months. Examples of bio-inert gels include polyethylene glycol hydrogels, polyvinyl alcohol hydrogels, alginate gels etc. In some embodiments, the polymeric precursor may also have active groups like aldehydes along its backbone or as end-groups that would enable cross-linking to the collagen in the trabecular and cortical bone thereby anchoring the bio-inert hydrogel to the surrounding bone.

The polymeric solution useful in embodiments of the present invention may also contain a radio-opaque agent to enable visualizing the location of the gel under fluoroscopy and to ensure that the inter-trabecular (femoral head, vertebral body, humeral head, etc.) region has been adequately filled with the gel. Alternatively, the polymeric backbone may be selected that is intrinsically radio-opaque. The radio-opaque agent may be attached to the polymeric backbone or could be mixed with the polymeric solution before it is cross-linked.

When employed in accordance with embodiments of the invention as described herein, a cross-linked hydrogel with low unconstrained compressive strength compared to cortical and trabecular bone, would be able to provide sufficient mechanical reinforcement when formed within the constraints of the cortical shell at the injection site. The compressive strength of cortical bone generally ranges from about 130-150 MPa (compressive modulus=15 GPa) and that of trabecular bone (cancellous bone) ranges from about 10 to 50 MPa (compressive modulus=1 GPa). The compressive strength of traditional bone cements range between about 5 and 400 MPa (compressive modulus=4 GPa) when measured in an unconstrained setting. Cortoss™, a cross-linked resin with glass-ceramic particles, has a compressive strength of 200 MPa and compressive modulus of 8 GPa. (Cortoss™ is a trademark of Orthovita Corporation). As another example, macroporous, injectable hardening resorbable calcium phosphate cements available from Graftys SA have a compressive strength of 12 MPa.

In exemplary embodiments of the present invention, the unconstrained compressive strength of cross-linked reinforcing gels would be less than about 5 MPa, more specifically less than about 1 MPa, and in some embodiments less than about 500 kPa. Additionally, the unconstrained compressive modulus of the cross-linked reinforcing gel in exemplary embodiments would be less than about 5000 kPa, more specifically less than about 2500 kPa, and in some embodiments less than about 1000 kPa. As used herein, unconstrained compressive strength refers to the compressive strength (failure load) measured by applying a uniaxial compressive load on the cross-linked gel without any constrains that limit the deformation of the gel in directions orthogonal to the direction of compression. Examples of unconstrained or unconfined mechanical compressive testing are described in Koob et al., Biomaterials, 24, p 1285-1292, 2003 and Browning et al., Journal of Biomedical Material Research A, 98A, 268-273, 2011.

In another aspect of exemplary embodiments of the present invention, the viscosity of the reinforcing gel prior to initiation of cross-linking at the time of injection into the target region would generally range from about 1 to about 5000 cp, more specifically from about 1 to about 1000 cp, and in some embodiments from about 1 to about 100 cp. As used herein, viscosity of the mixtures refers to viscosity measured at physiological temperature at low shear rates (zero shear viscosity). One advantage realized by embodiments of the present invention is that injecting a solution of low viscosity into the target region minimizes the pressure required to inject the solution and is less likely to disrupt the fragile trabecular bone at the treatment site (see FIGS. 3a-b). The cross-linked gel formed at the treatment site would surround the bony trabeculae as shown in the cross-sectional view in FIG. 3b.

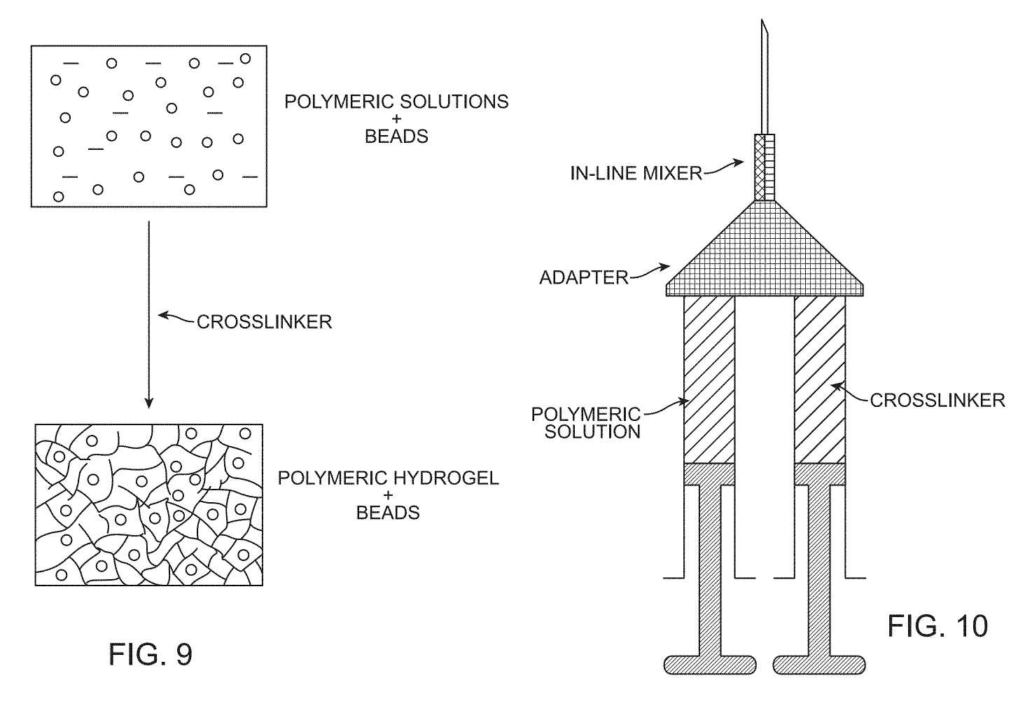

Compressibility of a material is the change in volume of a material when subjected to pressure or a compressive force. Compressibility is defined by its poisson's ratio. Poisson's ratio of a perfectly incompressible material is 0.5, with compressible materials having lower values. Based on theory, a material with high water content would have a poisson's ratio at or close to 0.5. The poisson's ratio of the cross-linked reinforcing gel according to embodiments of the present invention, in particular a hydrogel formed in-situ, may be lowered if desired for a particular application by mixing in additives. For example, beads which are not hydrophilic and have a poisson's ratio lower than 0.5 could be dispersed in the hydrogel to increase the compressibility of the composite hydrogel. To provide compressibility, it would be preferable for the beads to not draw and retain the water from the surrounding hydrogel. As an example, PMMA microspheres are considered to be compressible and have a poisson's ratio of less than 0.5. By adding PMMA microspheres to the aqueous polymeric solution prior to cross-linking as shown in FIG. 9, the composite hydrogel would have hydrophobic spheres dispersed in an aqueous environment thereby altering the compressibility of the resulting composite hydrogel. One skilled in the art would be able to optimize the composite hydrogel by varying the hydrophobicity of the beads/microspheres, concentration of the beads/microspheres, the size and polydispersity of the beads/microspheres, and the inherent compressibility of the beads/microspheres. The degradability of the beads/microspheres would ideally be similar to the surrounding aqueous hydrogel. In certain embodiments, the beads/microspheres may be sized to enable the composition to be injectable through a narrow gauge needle (smaller than 15 G) and disperse through the trabecular bone structure to ensure complete filling of the inter-trabecular space in the target bone. The viscosity of the polymeric solution with the hydrophobic beads/microspheres may be within the range disclosed above. To enable injecting a low viscosity polymeric solution into the target bone, the polymeric solution prior to injection may be substantially devoid of any particulate materials like calcium phosphate granules, hydroxyapatite granules, etc. The concentration (by weight or volume) of any particulate material would be less than about 15%, more specifically less than about 10%, most and in some embodiments less than about 5%.

In other exemplary embodiments, to confer compressibility to the cross-linked reinforcing gel, fat may be used as an additive that is mixed with the polymeric solution prior to cross-linking. The fat could be autologous, synthetic or allogenic. In one embodiment, the fluid contents of the femoral head or vertebral body may be aspirated out, and a portion of the aspirated material may be added to the polymeric solution prior to injecting the polymeric solution. The fluid contents may include red marrow, yellow marrow, fat, blood, etc. Alternately, the aspirated material may be separated to isolate the fat component, and then a portion or all of the fat component could be added to the polymeric solution. The volumetric ratio of the aspirate or fat added to the polymeric solution may be about 1:1, more specifically about 1:2, and in some embodiments about 1:4. In other embodiments, autologous fat may be aspirated from other bony sites (other than the injection site) or from non-bony tissue. Alternatively, allogenic fat aspirated from other individuals may be used. The aspirated fluid or fat may be mixed with the polymeric solution at the appropriate ratio prior to addition of the cross-linking component. Alternatively, the aspirate fluid or fat, polymeric solution and cross-linking component may be mixed simultaneously.

In further exemplary embodiments of the present invention, the cross-linking time may be less than about 2 hours, more specifically less than about 1 hour, and in some embodiments less than about 30 minutes. Cross-linking time is defined as the time required for at least 75% of the total cross-linking to be complete. For purposes of characterization, the degree of cross-linking may be determined using chemical methods, mechanical methods, thermal methods or any other means known in the art.

In one embodiment, the polymer and cross-linker are selected such that the reaction is not exothermic, and the temperature of the cross-linking mixture is substantially unchanged (not greater than 5° C. from its pre-cross-linked temperature) during the cross-linking period when measured in a controlled temperature environment. Not increasing the temperature of the surrounding bone during cross-linking reduces the risk of any deleterious effects on the surrounding bone.

The mixtures of reinforcing liquids in embodiments of the present invention may contain antibiotics, bone morphogenetic proteins, growth factors, cells, and other bioactive components. Gels, preferably hydrogels, can be selected such that they are biocompatible with bony tissue and allow the diffusion of nutrients to the cells, thereby not compromising the viability of the surrounding trabecular and cortical bone. The mixtures also may be formulated in solutions at acidic, basic or neutral pH and may contain buffer salts like phosphates, citrates, borates, etc.

The cross-linkable reinforcing liquids of embodiments of the present invention are injected in sterile form. The mixtures may be sterilized by sterile filtration through a sterilizing filter (for example, a 0.22 micron filter), by gamma and e-beam irradiation, by ethylene oxide or by moist heat. Other methods of sterilization acceptable in the medical device industry may also be used to sterilize the mixture. For sterilization purposes, the polymeric mixture and/or the cross-linking agent may be sterilized in a dry form (e.g., lyophilized powder) and then reconstituted at the surgical site at the time of use.

In other embodiments of the present invention, components of a system as described herein may be provided in various configurations. For example, the polymeric precursor and the cross-linker may be provided in a single container in a dry state such that it is hydrated at the time of use and injected immediately. Alternatively, the polymeric precursor and the cross-linker may be provided in separate containers in a dry state such that each is hydrated independently at the time of use and then mixed before use. Alternatively, either component could be provided in a pre-hydrated state. It would also be possible to mix one component in a hydrated state with the other component in a dry state. As would be obvious to one skilled in the art, there are a variety of delivery configurations all of which are considered to be within the scope of the invention.

The components could be mixed in a variety of volumetric ratios depending on a variety of factors such as the concentration of the components, the viscosity of the component solutions, the cross-linking time, etc. In one embodiment, the components are mixed in equal volumetric ratios for optimal mixing ease and efficiency.

The mixing of the components could be accomplished prior to injecting the mixture into the trabecular bone or during the injection, for example using a dual syringe with an in-line static mixer. Various devices and methods of mixing components for delivery are known in the medical device industry and may be adapted for use in embodiments of the present invention based on the teachings herein contained. Exemplary embodiments of devices that could be used to prepare the components, prepare the intra-osseous site, and deliver the materials, are described below.

FIG. 10 shows an exemplary embodiment of an injection device including a double barreled syringe with the polymeric solution in one barrel and the cross-linker in the other barrel delivered to the intra-osseous site through an in-line mixer. A Y-adapter may be used to transition from the syringes to the in-line mixer.

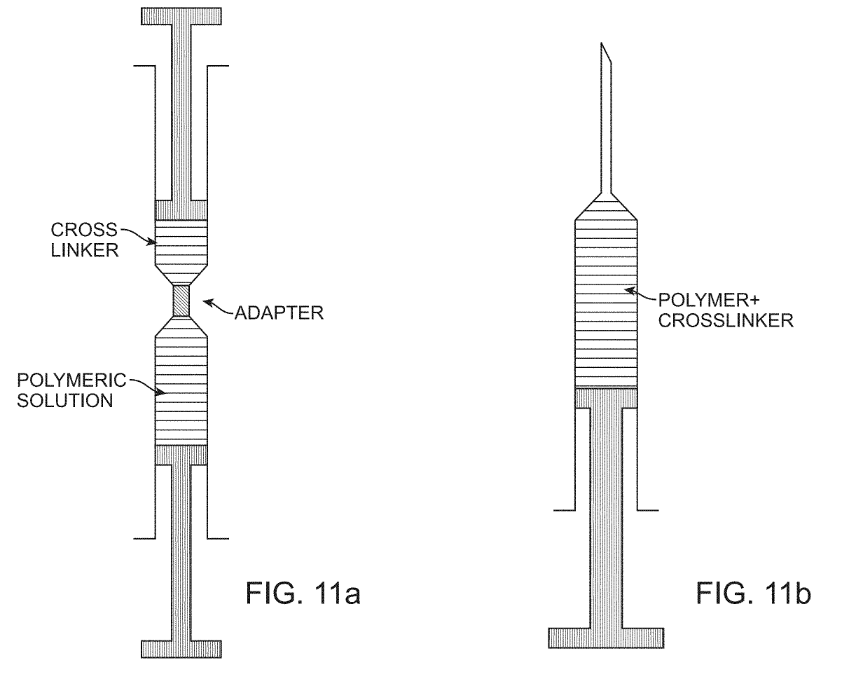

FIGS. 11a-b show a cross-linkable liquid mixture prepared according to an exemplary embodiment by injecting the cross-linker from one syringe to a second syringe containing the polymeric solution through an adapter. The mixture is then injected with the second syringe (FIG. 11b) through a needle into the intra-osseous site.

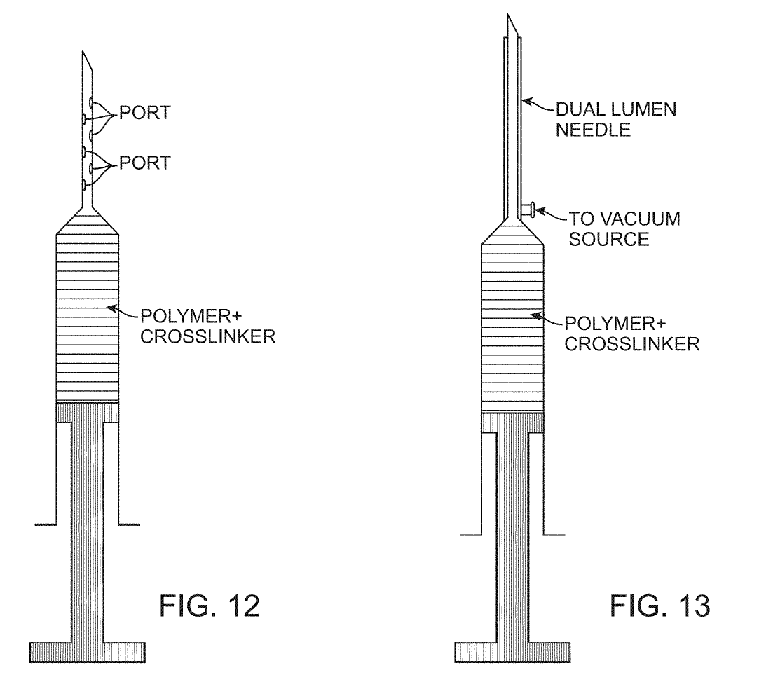

In another exemplary embodiment, as shown in FIG. 12, a mixture prepared as shown in FIG. 11 may be injected into the intra-osseous site through a needle with multiple ports along the sidewall of the needle to deliver the material to a larger region of the trabecular bone in a single injection.

FIG. 13 shows a mixture of cross-linkable reinforcing gel being delivered through a double lumen, coaxial needle syringe. In this embodiment, the outside lumen may be connected to a vacuum source (not shown) to aspirate residual material in the inter-trabecular space while the inner lumen is used to deliver the cross-linkable mixture.

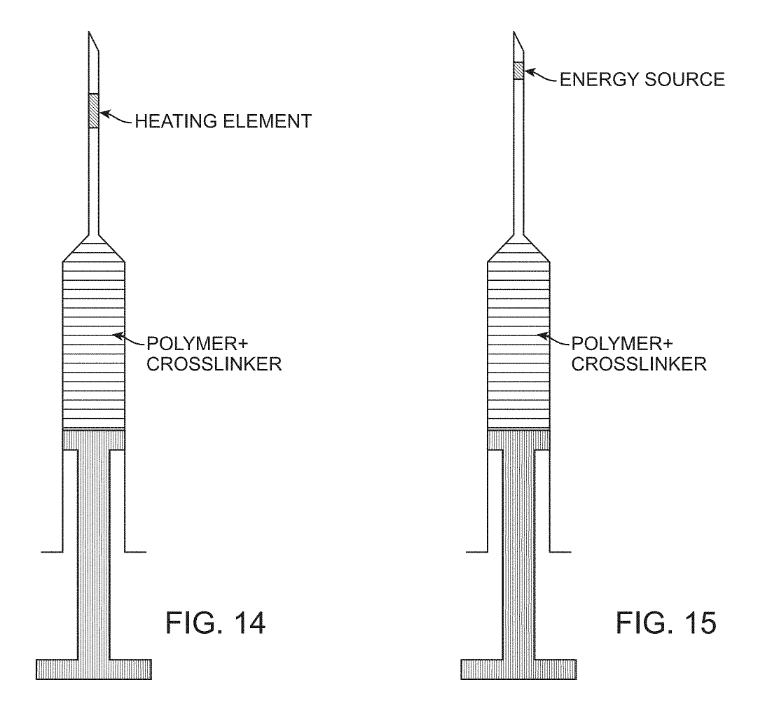

FIG. 14 shows a mixture of cross-linkable reinforcing gel being delivered through a syringe with an attached heating element which could be used to increase the local temperature in the trabecular bone to initiate or accelerate cross-linking. In this exemplary embodiment, the heating element could be at the tip of the needle, at the base of the needle or along the surface of the needle. The heating element may comprise a metallic electrode having a tubular sleeve-like shape with an attached wire that extends proximally along the needle and barrel of the syringe to a point where it can be coupled to a power source. If desired, the heating element may be electrically and/or thermally isolated from the remainder of the needle. The heating element may also be coupled to a separate probe which is placed into the trabecular bone region separately from the syringe, either before or after the polymeric solution and cross-linker have been delivered.

FIG. 15 shows another exemplary embodiment of an injection device with an ultrasound or microwave or other energy emitter at the tip that could be used to increase the local temperature to initiate or accelerate cross-linking. The energy emitter may comprise an ultrasound transducer, microwave antenna, radiofrequency electrode, or other suitable energy delivery means, and will be coupled to a lead or wire extending proximally along the needle and barrel of the syringe to a suitable coupling for connection to a generator or other energy source. The energy emitter may also be located at the proximal end of the needle or anywhere along the length of the needle.

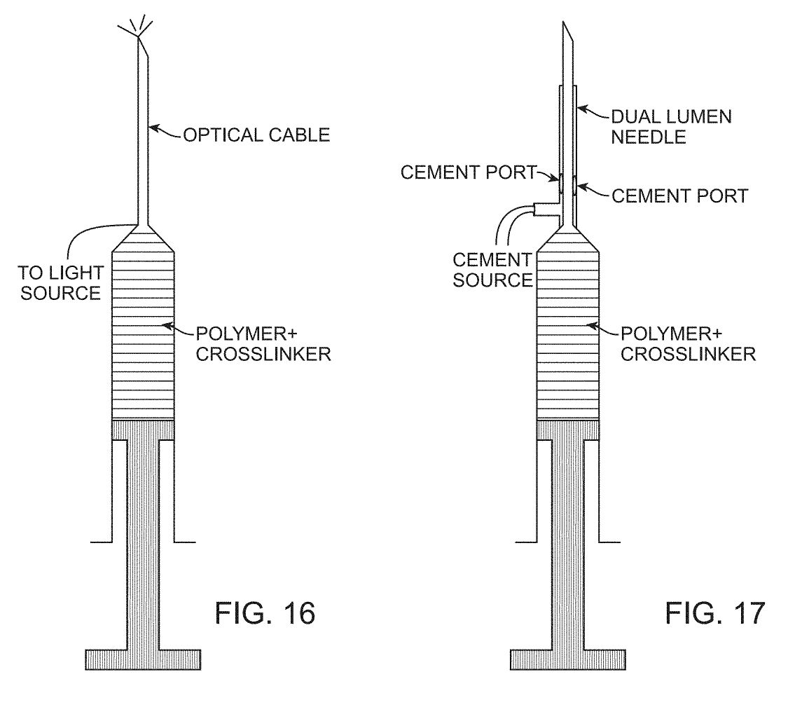

FIG. 16 shows a further exemplary embodiment of an injection device with an optical fiber to deliver optical energy (light) to initiate the cross-linking reaction.

FIG. 17 shows yet another exemplary embodiment of an injection device having a coaxial dual lumen needle having an outer lumen through which bone cement may be injected from an external source as a means to retain the hydrogel within a specific region of the trabecular bone, for example to create a bone cement plug or dam as previously described. The outer lumen may have ports in its sidewall through which the cement may be expelled into the bone. The inner lumen of the needle enables injecting the polymeric mixture into the trabecular bone. As with each of the injection devices described hereinabove, this exemplary device is based on a syringe comprising a barrel receiving a plunger to eject the liquid mixture. As will be appreciated by persons of ordinary skill in the art, other known injection type delivery devices may be employed, such as metering syringes or power actuated syringes, without departing from the teachings of the present invention.

The needles in the exemplary embodiments of injection devices described herein may include radio-opaque markers to enable visualization under fluoroscopy to target specific intra-osseous landmarks. The needles may also have temperature sensors, pressure sensors or other sensors to provide additional in-situ information to control the delivery of the polymeric mixture. Increases in pressure may be used to detect overfilling or device blockage while a sudden drop in pressure may be indicative of device leakage or leakage of the material outside the trabecular site. The plunger of the needle could be driven by a pressure source to assist in the injection, to ensure consistent flow of the polymeric mixture or to automatically stop the injection on achieving a pre-determined intra-osseous pressure. The devices and methods described above could be modified as required for each bony site (i.e., femoral head, spinal vertebrae, humeral head, etc.). Features from the various devices described above could be combined to design devices to address specific needs encountered for a particular clinical application. These devices are considered exemplary and a variety of modifications and additions could be made by one skilled in the art and are considered to be within the scope of this invention.

In some embodiments, the device for aspirating the fluid contents of the bony site could be a separate device. In other embodiments, the devices and components may be supplied in the form of a kit to enable performing the treatment procedure. The kit would typically include the polymeric component, the cross-linker and a delivery device. The kit may also include a trocar to achieve access into the intra-osseous location. If the components are provided in a dry form, the kit may include the appropriate buffer solutions. While the descriptions of containers have referred to syringes, other containers commonly used in the medical device industry like vials, ampules, cartridges, bottles, etc. may also be used to supply the components in the kit. The delivery device in the kit may contain an in-line mixer or a separate mixing apparatus to mix the components. The kit may also contain apparatus to solubilize the dry components in the appropriate buffers. When the delivery device includes sensors or requires external sources of power, energy, etc., the kit may include power cords, pressure tubes and other components to attach to the delivery device. The contents of the kit may all be sterile or just the components that are transferred into the sterile surgical field may be provided sterile.

The following prophetic examples further illustrate aspects and embodiments of the present invention:

### EXAMPLE 1

Mix 5 ml of 5% w/w PEODA (MW: 3.4 kDa) in phosphate buffered saline (PBS) with 100 μl of 1M ascorbic acid dissolved in DI water and 100 μl of 1M ammonium persulfate dissolved in DI water. Transfer the mixture into a closed cylindrical mold in a 37° C. water bath. Monitor the mixture in the tube for 30 minutes until a transparent gel forms.

### EXAMPLE 2

Mix 5 ml of 5% w/w PEOMA (MW: 3.4 kDa) in PBS with 100 μl of 1M ascorbic acid dissolved in DI water, 100 μl of 1M ammonium persulfate dissolved in DI water and 100 μl of 1M FeCl₃ dissolved in DI water. Transfer the mixture into a closed cylindrical mold in a 37° C. water bath. Monitor the mixture in the tube for 30-60 minutes until a transparent gel forms.

### EXAMPLE 3

Mix 5 ml of 5% w/w PEODA (MW: 3.4 kDa) in PBS with 10 μl of 0.01M glucose oxidase dissolved in PBS, 100 μl of 0.01M ferrous sulfate dissolved in PBS and 50 μl of 0.5M of glucose dissolved in PBS. Transfer the mixture into a closed cylindrical tube in a 37° C. water bath. Monitor the mixture in the tube for 30-60 minutes until a transparent gel forms.

### EXAMPLE 4

Mix 5 ml of 5% w/w PEODA (MW: 3.4 kDa) in phosphate buffered saline (PBS) with 100 μl of 1M ascorbic acid dissolved in DI water, 100 μl of 1M ammonium persulfate dissolved in DI water and 10 μl of 0.1M of sodium iothalamate (contrast agent) dissolved in DI water. Transfer the mixture into a closed cylindrical mold in a 37° C. water bath. Monitor the mixture in the tube for 30-60 minutes until a transparent gel forms.

### EXAMPLE 5

Obtain an osteoporotic vertebral body. Place a 15 G needle connected to a 5 cc syringe into the vertebral body through the pedicle. Aspirate about 2 ml of marrow fluid. Mix 2 ml of the marrow aspirate with 20 ml of 5% w/w PEODA (MW: 3.4 kDa) in phosphate buffered saline (PBS) with 400 μl of 1M ascorbic acid dissolved in DI water, 400 μl of 1M ammonium persulfate dissolved in DI water and 40 μl of 0.1M of sodium iothalamate (contrast agent) dissolved in DI water. Transfer the mixture into a closed cylindrical mold in a 37° C. water bath. Monitor the mixture in the tube for 30-60 minutes until a gel forms.

### EXAMPLE 6

Obtain an osteoporotic femur. Place a 15 G needle connected to a 5 cc syringe into the femoral head through the greater trochanter. Aspirate about 4 ml of marrow fluid. Mix 4 ml of the marrow aspirate with 40 ml of 5% w/w PEODA (MW: 3.4 kDa) in phosphate buffered saline (PBS) with 800 μl of 1M ascorbic acid dissolved in DI water, 800 μl of 1M ammonium persulfate dissolved in DI water and 80 μl of 0.1M of sodium iothalamate (contrast agent) dissolved in DI water. Transfer the mixture into a closed cylindrical mold in a 37° C. water bath. Monitor the mixture in the tube for 30-60 minutes until a gel forms.

### EXAMPLE 7

Obtain an osteoporotic lumbar vertebral body. Aspirate all the marrow content of the vertebral body using a 15 G needle. Prepare 20 ml of hydrogel mixture as described in Example 5. Inject the mixture into the vertebral body through an 18 G needle under fluoroscopic visualization until the contrast agent in the hydrogel is visible across the entire vertebral body. If necessary, obtain fluoroscopic view from two orthogonal directions to confirm that the hydrogel is completely filling the vertebral body. Incubate the vertebral body for at least 15 minutes at 37° C. Remove the vertebral body and section it with a saw to visually confirm that the hydrogel fills the entire vertebral body.

### EXAMPLE 8

Obtain an osteoporotic femur. Aspirate all the marrow content of the femoral head and neck using a 15 G needle. Prepare 40 ml of hydrogel mixture as described in Example 6. Inject the mixture into the femoral head/neck through an 18 G needle under fluoroscopic visualization until the contrast agent in the hydrogel is visible across the entire femoral head and neck. Move the needle while injecting the mixture to ensure it fills the femoral head/neck uniformly. If necessary, obtain fluoroscopic view from two orthogonal directions to confirm that the hydrogel is completely filling the femoral head/neck. Incubate the femur for at least 15 minutes at 37° C. Remove the femur and section it with a saw to visually confirm that the hydrogel fills the entire femoral head/neck.

### EXAMPLE 9

Reinforce an osteoporotic vertebral body by filling it with a hydrogel as described in Example 7. Measure the fracture strength of the reinforced vertebral body as described by Bal et al., 45^{th} Annual Meeting, Orthopedic Research Society, February 1999. Compare the fracture strength to the fracture strength of an unreinforced osteoporotic vertebral body.

### EXAMPLE 10

Reinforce an osteoporotic femur by filling the femoral head/neck with a hydrogel as described in Example 8. Measure the fracture strength of the reinforced femur as described by Beckman et al., Medical Engineering and Physics, 29, 755-764, 2007. Compare the fracture strength to the fracture strength of an unreinforced contralateral femur.

### EXAMPLE 11

Obtain a lumbar vertebral segment (two vertebrae with the intervening intervertebral disc). Perform discography as described by Heggeness et al., Spine, 18, p 1050-1053, 1993, to measure the end plate defection of the adjacent end plates. Reinforce one of the vertebral bodies with a hydrogel as described in Example 7. Repeat discography and measure end plate deflection of the adjacent end plates.

While the invention has been illustrated by examples and descriptions of aqueous systems and hydrogels, it will be understood that the invention may also have application with any biocompatible incompressible fluid.

It is well known that fracture risk is high in certain patient groups like the elderly, patients on long-term steroid therapies, patients with kidney disease, etc. New models are being developed to improve the predictability of fracture risk (e.g., FRX). The method described in the present invention could be used as adjunct therapy to treat adjacent vertebral bodies in high risk patients undergoing kyphoplasty, vertebroplasty or spinal fusion where there is a high risk of adjacent vertebral body fracture. Similarly, adjunct therapy could be prescribed to treat the contralateral hip in high risk patients undergoing treatment for a primary hip fracture.

In the descriptions and examples provided here, the methods and devices are intended to be illustrative, and variations may be made by one skilled in the art. It is intended that such modifications, changes and substitutions are included in the scope of the invention as set forth in the following claims.

1. A method for reinforcing endplates of a vertebral body, the vertebral body including a region of trabecular bone, the method comprising:
delivering an aqueous solution of a non-cross-linked or substantially non-cross-linked polymer into the region of trabecular bone in the vertebral body, wherein the polymer cross-links in-situ to form a non-degradable hydrogel.

2. The method according to claim 1, wherein the hydrogel fills at least 85% of a volume of the vertebral body.

3. The method according to claim 1, wherein the delivering step reduces a deflection of at least one of the endplates by at least 50%.

4. The method according to claim 3, wherein the method is used for treating back pain.

5. The method according to claim 1, further comprising evacuating the vertebral body prior to the delivering step.

6. The method according to claim 1, wherein the polymer is radio-opaque.

7. The method according to claim 1, wherein the polymer is free of hydrophobic domains.

8. The method according to claim 1, wherein cross-linked hydrogel has a compressive modulus of less than 5000 kPa.

9. A method for reducing vertebrogenic back pain, comprising: delivering an aqueous solution of a non-cross-linked or substantially non-cross-linked polymer into a region of trabecular bone in a vertebral body, wherein the polymer cross-links in-situ to form a non-degradable hydrogel.

10. The method according to claim 9, further comprising aspirating a material out of the vertebral body prior to the delivering step.

11. The method according to claim 10, wherein the hydrogel fills at least 85% of a volume of the vertebral body.

12. The method according to claim 9, wherein the delivering step does not alter a trabecular bone structure in the vertebral body.

13. The method according to claim 9, wherein the aqueous solution of a non-cross-linked or substantially non-cross-linked polymer further includes a radio-opaque agent.

14. The method according to claim 9, wherein the hydrogel is bioinert.

15. The method according to claim 9, wherein the polymer is free of hydrophobic domains.

16. The method according to claim 9, wherein cross-linked hydrogel has a compressive modulus of less than 1000 kPa.

17. A method for reinforcing a bone having a region of trabecular bone surrounded at least in part by a layer of cortical bone, the method comprising:
delivering an aqueous solution of an at least substantially non-cross-linked, hydrophilic polymer free of hydrophobic domains into the region of trabecular bone; and
cross-linking the polymer in situ to form a non-degradable hydrogel with a compressive modulus of less than 5000 kPa.

18. The method in claim 17, wherein said delivering comprises filling substantially more than half of the volume of interstices defined by the region of trabecular bone with said aqueous solution.

19. A method for reinforcing a bone having a region of trabecular bone with a trabecular structure, surrounded at least in part by a layer of cortical bone, the method comprising: injecting an aqueous polymeric solution into the trabecular bone, and filling substantially more than half of the volume of interstices defined by the region of trabecular bone with said aqueous solution, such that the polymer cross-links in situ to form a non-degradable hydrogel having a compressive modulus of less than about 1000 kPa, wherein the trabecular structure is not substantially altered.

20. The method in claim 19, wherein the bone marrow is aspirated out prior to injecting the aqueous polymeric solution.
